(19) United States
(12) Patent Application Publication
(10) Pub. No.: US 2007/0191756 A1
(43) Pub. Date: Aug. 16, 2007

(54) System, method and apparatus for enhanced sweat control and the like

(76) Inventor: Robert Tapper, Los Angeles, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/651,930

(22) Filed: Jan. 10, 2007

Related U.S. Application Data
(60) Provisional application No. 60/772,763, filed on Feb. 13, 2006.
(60) Provisional application No. 60/833,528, filed on Jul. 25, 2006.
(60) Provisional application No. 60/850,487, filed on Oct. 10, 2006.

(51) Int. Cl. A61N 1/30; F26B 7/00

(52) U.S. Cl. 604/20; 34/389

(57) ABSTRACT

This invention provides aluminum or aluminum alloy spheres/particles or a mixture of aluminum and aluminum alloy and/or aluminum zirconium powder to form an electrode together with an aluminum metal screen wire mesh or the like within a membrane pouch and in contact with an electrical power source. The heavy metal particles are combined with a salt water or gel solution. The membrane enclosure includes a wire mesh which is surrounded by a quantity of the aluminum micro-sized particles. Magnesium and/or copper particles may also be utilized. The porous membrane enclosure has an opening through which a wire is placed in contact with the electrical power source. The contact between the power source terminal and membrane enclosure either physically touches each other or the power source directly touches the internal wire mesh through a small opening in the membrane enclosure. The power source can be a DC supply. The system provides long term sweat control. The system may be wetted with tap water, distilled water and a saline gel solution mixed with the aluminum particles as the electrically conductive medium between the power source and the skin. A deodorant or fragrance is added to the long term sweat control system and exhibits characteristics lasting much longer than topical application.

### CROSS-REFERENCES TO RELATED APPLICATIONS

This application is claiming the benefit of co-pending provisional application No. 60/772,763 filed on Feb. 13, 2006; provisional application No. 60/833,528 filed on Jul. 25, 2006 and provisional application No. 60/850,487 filed on Oct. 10, 2006. The following documents are incorporated herein by reference: United States Publication No. U.S. 2005/0192528 A1, Publication Date Sep. 1, 2005 entitled METHODS, APPARATUS AND CHARGED CHEMICALS FOR CONTROL OF IONS, MOLECULES OR ELECTRONS; United States Publication No. U.S. 2006/0025714 A1, Publication Date Feb. 2, 2006 entitled METHODS, APPARATUS AND CHARGED CHEMICALS FOR CONTROL OF IONS, MOLECULES OR ELECTRONS.

This invention relates generally to sweat control and improvements in related electrotherapeutic systems and, more particularly, to improved long term sweat control systems utilizing small aluminum particles spheres and the like which increase electrical current flow, enhance long term sweat control and also prevent skin injury in a sweat control device.

### BACKGROUND OF THE INVENTION

A problem with present electrotherapeutic devices intended for long term sweat control is that the solid metal sheet electrode from which the signal emanates, invariably causes the signal to be emitted nonuniformly from the positive electrode. This results in “hot spots” (of hydrochloric acid) at the delivery site often resulting in skin injury. A new electrode included in the present invention evenly disperses the signal and neutralizes the hydrochloric acid to prevent injury or irritation. This new electrode equally applies to all therapeutic modalities including iontophoresis and stimulation devices.

### SUMMARY OF THE INVENTION

Briefly, and in general terms, the present invention provides aluminum or aluminum alloy spheres/particles/powder or a mixture of aluminum and aluminum alloy and/or aluminum zirconium powder to form an electrode together with an aluminum metal screen wire mesh or the like within a membrane pouch and in contact with an electrical power source. The heavy metal particles are combined with a salt water or gel solution. The membrane enclosure includes a wire mesh which is surrounded by a quantity of the aluminum micro-sized particles. Magnesium or zinc particles may also be utilized. This provides a positive polarity electrode for the system.

The porous membrane enclosure has an opening through which a wire is placed in contact with the electrical power source. The contact between the power source terminal and membrane enclosure either physically touches each other or the power source directly touches the internal aluminum wire mesh through a small opening in the membrane enclosure. The power source may be a DC supply. The resulting system provides long term sweat control.

The above system may be wetted with tap water, distilled water and a saline gel solution mixed with the aluminum particles as the electrically conductive medium between the electrical power source and the skin.

An additional major objective of this invention is the prevention of skin irritation by the hydrochloric acid generated at the positive pole electrode. The aluminum particles reformulate the irritation causing hydrochloric acid to nullify the potential for injury.

With the negative return electrode, stainless steel particles are used surrounding a stainless steel wire mesh sealed within a porous membrane pouch. The stainless steel mesh attaches to the negative battery (DC) polarity through a small opening in the pouch. The stainless steel particles which are positively charged, offset the injury-causing sodium hydroxide emanating from this electrode. Alternative means of stopping the injury-causing sodium hydroxide are the use of charged membranes, ion exchange resins or other buffers.

It is the expectation of consumers that the OTC (over the counter) antiperspirant include a deodorant or fragrance. When a deodorant or fragrance is added to the long term sweat control system described above, it too will exhibit long term characteristics giving it a major advantage over present OTC products. Applied in this manner, the deodorant or fragrance would last longer than mere topical application.

Another object is to provide a stand-alone drug delivery patch that includes a fragrance (and other beneficial drugs or skin conditioning agents or fillers) and a powdered aluminum electrode or the like for a long lasting fragrance or other beneficial product to be applied anywhere on the human body.

These and other objects and advantages of the invention will become apparent from the following more detailed description, when taken in conjunction with the accompanying drawings of illustrative embodiments.

### BRIEF DESCRIPTION OF THE DRAWINGS

FIG. 1 is an exploded perspective view of a presently preferred embodiment of a sweat control device constructed in accordance with the present invention;

FIG. 1a is a perspective view of an underarm treatment device constructed in accordance with the invention and shows a pouch covered with a wetted pad;

FIG. 1b is an exploded view of the pouch containing an aluminum screen and powdered aluminum;

FIG. 1c is an exploded view of the open pouch displaying an aluminum screen and aluminum powder;

FIG. 1d is an enlarged exploded view of the aluminum screen and powdered aluminum;

FIG. 1e is a perspective view illustrating a hand return unit with electronic signal, battery and thumb wheel control;

FIG. 2 is an electrical schematic of circuitry suitable for the device shown in FIG. 1; and

FIG. 3 is an isometric view of one form of a shoulder unit which may be provided in accordance with an alternative embodiment of the invention.

### DETAILED DESCRIPTION OF THE PREFERRED EMBODIMENTS

The use of aluminum particles/powder contained in a pouch constitutes a novel positive polarity electrode. The positive electrode's advantages are that it changes injury causing hydrochloric acids to aluminum chloride and other forms, all of which are identified as antiperspirants. It also vastly increases current, which acts to speed up the sweat control process. The powder is formulated with a saline gel.

The negative return electrode uses stainless steel powder, disperses injury causing sodium hydroxide, and also allows for a very large increase in current. This steel powder is also formulated with a saline gel. Other means of rendering sodium hydroxide harmless may include charged membranes, ion exchange resins and other buffers.

A solid aluminum plate used in an iontophoretic device for long term sweat control using tap water, has been found to generate hydrochloric acid and cause an uneven delivery of current. This can result in skin injury. Recognizing this, the use of aluminum mesh has been suggested for more even distribution of signal over the treatment area. This invention concerns a still further improvement over the mesh by a vast increase of surface area through the use of micro-sized aluminum particles, spheres or the like. Such aluminum particles may be obtained, for example, from Valimet, Inc., 431 Sperry Road, Stockton, Calif. 95206. The aluminum could be an alloy of zirconium, or zirconium particles may represent a small percentage of the particles with aluminum particles occupying the balance. These particles may range from micron to nano in size. The aluminum particles, therefore, replace the metal sheet or plate that was previously used to transmit a signal into the skin. It has been discovered that the aluminum particles that surround the wire mesh to which the positive polarity is connected, cause the beneficial effect of neutralizing the hydrochloric acid generated at the wire mesh. This occurs by chemically converting the hydrochloric acid to aluminum chloride hydroxide. Injury producing sodium hydroxide ions emanating from the negative electrode are prevented from reaching the skin by using positively charged stainless steel particles contained in a porous membrane as the electrode. Other methods of stopping sodium hydroxide may be done by using charged membranes, ion exchange resins or other buffers.

The phenomena associated with long term sweat control can have another and important use with a long lasting deodorant/fragrance/perfume. Literature describes this phenomena as parakeratotic plugs developed in the eccrine sweat duct caused by the electric current traversing the sweat duct. Previously, a fragrance could last maximally up to 18 hours. With the present invention the fragrance can last many days duplicating the longevity of sweat control for many weeks. The same technology can be applied to skin conditioning agents or other chemicals or drugs for ‘storage’ in the eccrine sweat duct for slow release. For instance, moisturizer creams can give continuous beneficial effects to the skin over time. Drugs for certain problems could also medicate continuously over time in a stand-alone delivery system.

Again, the invention can also be provided as a stand-alone drug delivery patch that includes a fragrance (and other beneficial drugs or skin conditioning agents or fillers) and a powdered aluminum electrode or the like for a long lasting fragrance or other beneficial product to be applied anywhere on the human body.

The device is typically powered by two 12 volt alkaline batteries in series for a total of 24 volts and capable of delivering up to 7 ma or more. The above application requires as a power source a DC supply and other suitable battery configurations may be used without departing from the invention. Shock and pain prevention may be further enhanced by slow ramp up and/or down of the applied electrical current (e.g., see U.S. Pat. No. 4,340,047).

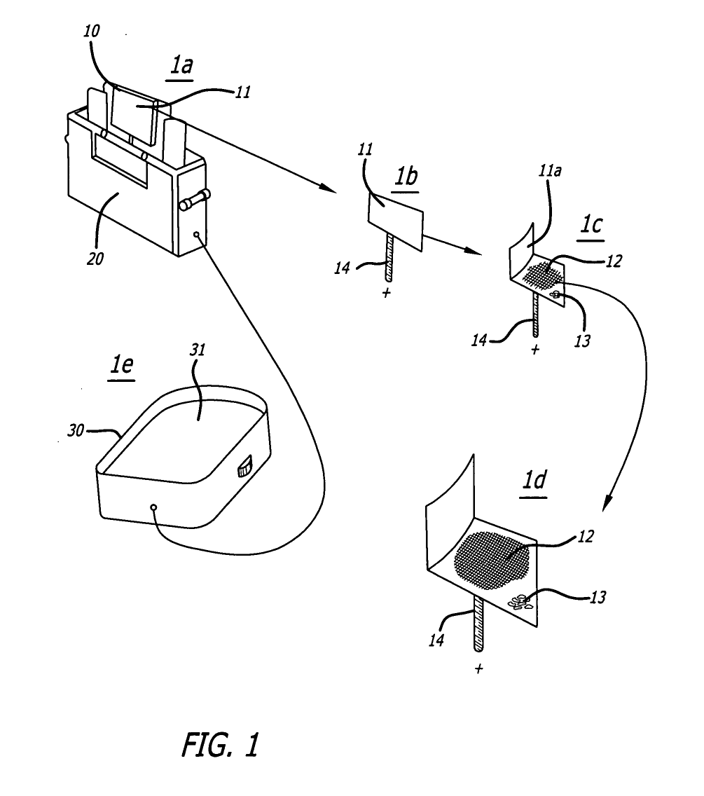

As best observed in FIG. 1 and FIGS. 1a-1d, a bag 10 of aluminum or aluminum compound or alloy particles 13 is contained within a pouch 11 made of at least one layer of a porous membrane 11a. As shown in FIG. 1a, the pouch 11 is covered with a wetted pad. As seen in FIGS. 1c and 1d, the particles rest on a metal or other electrically conductive mesh 12 or the like (such as an aluminum screen) within the pouch, the mesh and therefore the particles being in electrical contact with a conductive lead 14, typically aluminum, preferably in a DC situation connected to a source (a battery or the like) of positive potential. The aluminum screen is surrounded by fine powdered aluminum and the pouch enclosure is a porous membrane. The pouch is then surrounded by a wetted felt intervenor or water vessel equivalent in the housing 20 (FIG. 1a) to provide electrical conductivity between the skin and the battery terminal.

A hand return unit 30 is shown in FIG. 1e and shows a device for electronic signal, battery and control and is electrically connected to the device 20 in FIG. 1a. The hand return unit 30 has a tap water saturated pad 31. Underneath the pad 31 is a stainless steel powdered electrode or charged membranes or a buffer to prevent skin injury.

It has also been discovered that magnesium particles may be utilized either alone or in combination with aluminum particles for sweat inhibition. Copper particles may also be utilized and provide further antifungal benefits. Zinc particles may also be used.

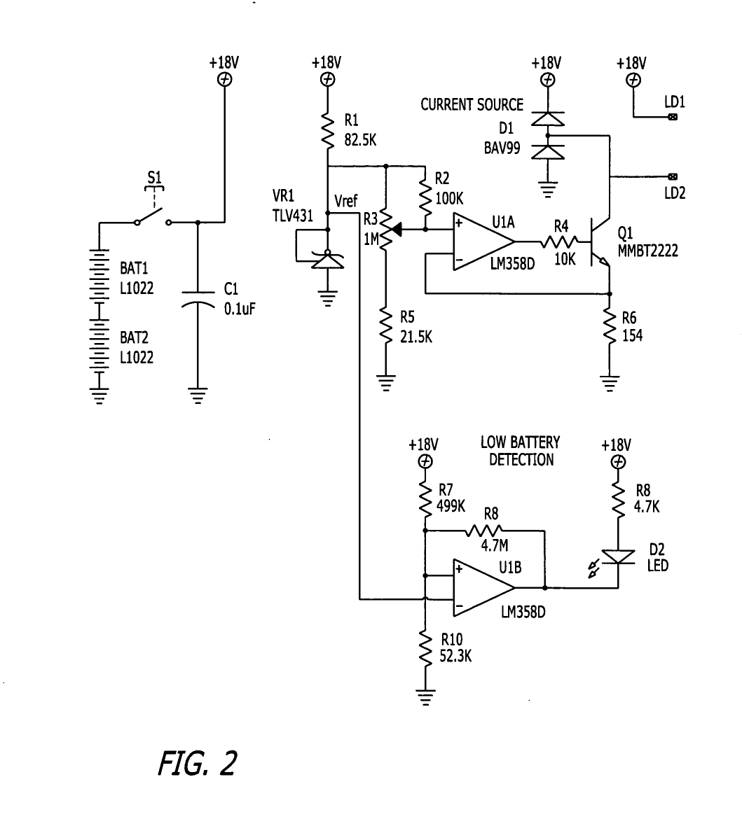

Referring to FIG. 2, there is shown an electrical schematic design of a presently preferred embodiment of an electrical circuit suitable for practice of the invention.

As shown in FIG. 2, S1 mechanically controls R3. When turned on, the battery will supply the +24 bus. R1 is chosen to delivery a minimum of 120 uA into the reference node Vref. VRI which will provide a stable Vref of 1.24V. A fraction of the current will be deviated by the circuitry around R3 and the value of R1 must guarantee a cathode current share of at least 100 uA for VR1 as the TLV431 would otherwise fall out of regulation. This minimum current must be maintained down to the lowest allowed battery voltage.

R3 is a linear potentiometer or membrane switches. Parallel resistor R2 causes R3 to deviate in a non-linear pattern so that larger voltage values and thus larger load currents occupy a broader wiper range. This increases resolution for larger current values at the expense of resolution for small currents. Changing R2 will alter the non-linear pattern, the smaller R2 the more non-linear. However, it is important to recalculate the reference current margin and if necessary adjust the value of R1. The ratio R2/R5 determines the ratio between minimum and maximum settings, and thus load currents. Leaving R2 off and re-calculating R5 would create a linear relationship between potentiometer and load current if desired.

Log-taper potentiometers or versions with lower resistance can be used as well. However, the lower the resistance the more current needs to be provided across R1 and that will reduce battery lifetime.

More elaborate non-linearities can be introduced via a resistor diode ladder. This will require more circuit board space and is commonly called piece-wise linear approximation.

U1A and Q1 form a voltage controlled current source. R6 equals 1.24 V/Imax. D1 provides ESD protection. U1B monitors the battery voltage against Vref. R9 and provides a weak Schmitt hysteresis so that the LED D2 remains on during minor load fluctuations after a low voltage has been signaled. R8 should be chosen so that the LED sees its specified current at the selected low battery warning voltage. With the current values listed in FIG. 2 for R7, R9 and R10, low voltage is signaled below 12V.

The circuitry consumes about 1 mA while the under-voltage LED is not lit. Most of that current is due to the quiescent draw of the two opamps U1A, U1B and can be reduced by using other devices. However, in general lower power devices will cost more.

The LM358 is functionally equivalent to the LM324 but smaller. An LM324 can be used for experiments. In that case the two unused opamp sections shall have their IN+ grounded and IN− tied to OUT. For the utmost in power savings an opamp from the LP Series could be chosen at higher cost. Another option to save space is the use of two separate opamps in SOT23-5 packages.

Additional circuitry could include a current level indicator using LEDs. This circuit monitors the voltage across a resistor in series with the load. Three LEDs are illuminated, one at a time, to signal three current ranges. Diodes shut off the lower level indicators as soon as the next higher range has been reached. To reduce current drain on the battery, a strobe oscillator flashes the LEDs by toggling the LED return node.

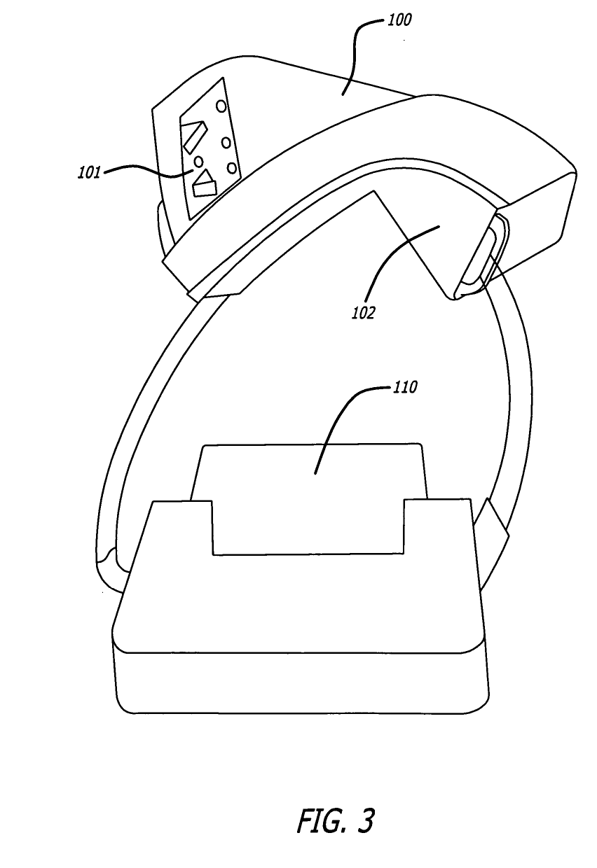

FIG. 3 shows another alternative embodiment of a system which may be constructed in accordance with the invention and having a negative return on the shoulder model. The figure is essentially self-explanatory. An electric housing 100 includes an operator panel 101. A negative return electrode 102 is placed on top of the shoulder underneath this wetted felt pad and surrounding negative electrode (not shown) are charged membranes 102. A wetted pad 110 and positive active electrode are also provided for treating the underarm, and underneath the wetted felt pad is an aluminum particle pouch in direct contact with the positive electrode (not shown).

It is the expectation of consumers that the OTC (over the counter) antiperspirant include a deodorant or fragrance. When a deodorant or fragrance is added to the long term sweat control system described above, it too will exhibit long term characteristics giving it a major advantage over present OTC products. Applied in this manner the deodorant or fragrance would last longer than topical application.

The application also allows for the infusion of a fragrance in keeping with the market expectation of a deodorant with antiperspirant. The present invention provides a long lasting fragrance since the aluminum particles described above act to create a long lasting antiperspirant. The use of a fragrance combined with the aluminum will also last exceptionally long.

Obviously, other skin improvement products such as moisturizing cream may be substituted for the perfume and new skin-improvement benefits will result because of the long lasting effect of the driving powdered aluminum electrode, independently of any antiperspirant application.

It will be apparent from the foregoing that, while particular forms of the invention have been illustrated and described, various alternatives, modifications and variations can be made without departing from the spirit and scope of the invention. Accordingly, the invention is intended to embrace all such alternatives, modifications and variations and it is not intended that the invention be limited, except as by the appended claims.

I claim:

1. In a system for sweat control in a biological subject, the improvement comprising:
a plurality of aluminum and/or aluminum alloy particles;
a source of electrical current; and
means for electrically applying said source to said particles and to the subject whereby sweat inhibition is achieved in the subject being treated.

2. A combination as recited in claim 1 wherein said micro-particles are micro-spheres.

3. A combination as recited in either of claims 1 or 2 wherein said source of electrical current is DC.

4. A system as set forth in any of claims 1-3 embodying a first electrode including aluminum micro-spheres.

5. A system as set forth in claim 4 wherein said micro-spheres range in size from micron to nanos.

6. A system as set forth in any of claims 1-5 embodying a second electrode comprised of stainless steel particles and a stainless steel wire screen contained within a porous membrane.

7. A positive active therapeutic electrode, comprising:
a plurality of aluminum and/or aluminum alloy particles;
a source of electrical current; and
means for electrically applying said source to said particles and to the subject.

8. A combination as recited in claim 7 wherein said micro-particles are micro-spheres.

9. A combination as recited in either of claims 7 or 8 wherein said source of electrical current is DC.

10. A negative therapeutic electrode comprising:
stainless steel particles mixed with a saline or gel solution.

11. A sweat control system, comprising:
a source of electric current;
a controller;
electrode means connected to said controller;
a plurality of aluminum and/or aluminum alloy particles as said electrode means; and
means for sending an electrical signal to a treatment site.

12. A system as set forth in claim 7, wherein said electrical current is DC.

13. A system as set forth in any of claims 1-9 wherein said particles further include an aluminum-based compound.

14. A system as set forth in any of claims 1-9 wherein the compound may consist of one of either aluminum or aluminum alloy.

15. A system and/or method as recited in any of the previous claims and further including:
a membrane pouch enclosing said aluminum/aluminum alloy particles acting as an electrode within said pouch.

16. A system and/or method as recited in claim 15 wherein a wetted felt pad or water vessel equivalent surrounds said pouch.

17. In an electrotherapeutic system, the improvement comprising:
aluminum and/or aluminum alloy particles in powder form in contact with an electrical power source.

18. A system as set forth in claim 17, wherein said electrical power source is DC.

19. In a sweat control system, the improvement comprising:
aluminum zirconium spheres and aluminum wire mesh contained in a small porous membrane enclosure.

20. In a sweat control system, the improvement comprising:
a membrane enclosure including a wire mesh upon which is placed a quantity of aluminum micro-sized particles.

21. The improvement recited in claim 20 and further comprising:
an electrical power source;
membrane enclosure being in contact with said power source; and
a power source terminal made of aluminum; and wherein the contact between said power source terminal and said membrane enclosure either physically touches each other or the power source directly touches an internal wire mesh through a small opening in the membrane enclosure.

22. A system as recited in claim 21 wherein said electrical power source is DC.

23. For use in a sweat control system as recited in any of the foregoing claims, the improved method comprising:
wetting the system with tap water or distilled water and a saline gel solution mixed with the aluminum particle electrode as an electrically conductive medium between the electrical power source and the skin of a subject being treated.

24. A sweat control system as recited in any of the foregoing claims, and further including:
a conductive gel mixed with aluminum spheres having a saline concentration of approximately six percent to increase electrical power efficiency.

25. A sweat control system as recited in claim 24, wherein DC electrical current is utilized.

26. A sweat control system as recited in any of the foregoing claims wherein magnesium is utilized alone or in combination with aluminum particles.

27. A therapeutic method and/or system as set forth in any of the foregoing claims, wherein an improved negative electrode comprises:
stainless steel particles and a stainless steel wire mesh contained within a porous membrane; and
a stainless steel wire emerging from the pouch for connection to a DC electrical source.

28. A method of sweat control wherein magnesium particles are utilized alone or in combination with aluminum particles for sweat inhibition.

29. A sweat control method and/or system as recited in any of the foregoing claims including means for electrically delivering a deodorant/fragrance/perfume, skin conditioning agents or chemical or drug in addition to accomplishing sweat control.

30. A system as recited in any of the foregoing claims including means for electrically delivering a deodorant/fragrance/perfume, skin conditioning agents or chemical or drug.

31. In a system for applying long lasting deodorant/fragrance/perfume, skin conditioning agents and/or any other chemical or drug to a biological subject, the combination comprising:
a system source of deodorant/fragrance/perfume, skin conditioning agents, chemicals, drugs or the like;
an electrode composed of a plurality of aluminum and/or aluminum alloy particles;
a source of electrical current;
means for electrically applying said source of current to said system source and to the subject whereby infusion of said deodorant/fragrance/perfume, skin conditioning agents or any beneficial chemical or drug is achieved in the subject being treated.

32. A combination as recited in claim 31 wherein said source of electrical current is DC.

33. In a system for delivery of long term fragrance, skin conditioning agents or any other beneficial chemical or drug to a subject, the combination comprising:
aluminum chlorohydrate or equivalent antiperspirant as the deodorant solution;
an active fragrance or chemical or drug; and
an additional means of mitigating injury causing ions selected from the group comprising stainless steel particles, charged membranes, and silver, silver/chloride electrodes.

34. In a therapeutic system, a positive active electrode comprising particles from the group comprising:
individually and/or combination aluminum, aluminum alloy, zirconium, zinc, copper, magnesium, whereby the effects of hydrochloric acid injury are mitigated.

35. In a therapeutic system a negative (return) electrode comprising:
stainless steel particles whereby the effects of sodium hydroxide and the like are mitigated.

36. A long lasting fragrance comprising:
aluminum electrode particles; and a fragrance combined with said aluminum particles.

37. For use in the application of long lasting fragrance, the combination comprising:
metal particles from the group comprising aluminum, aluminum alloy, zirconium and zinc;
a fragrance mixed with said particles; and
electrical means for applying the mixture to a surface being treated.

38. A stand-alone drug delivery patch for delivering fragrance, skin conditioning agents, fillers or any other beneficial chemical or drug to a subject, comprising:
a powdered aluminum electrode; and
an active fragrance or chemical or drug adapted to be driven by said electrode for application anywhere on the human body.

39. Each and every novel feature and/or novel combination of features herein disclosed.
